(19) United States
(12) Patent Application Publication
(10) Pub. No.: US 2005/0186269 A1
(43) Pub. Date: Aug. 25, 2005

(54) Stabilized feverfew formulations

(76) Inventors: Ronald G. Udell, Beverly Hills, CA (US); Roy Bouskila, Galilee (IL)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/058,069

(22) Filed: Feb. 15, 2005

Related U.S. Application Data
(60) Provisional application No. 60/547,528, filed on Feb. 25, 2004.

(51) Int. Cl. A61K 35/78; A61K 31/525; A61K 9/54; A61K 33/08

(52) U.S. Cl. 424/458; 424/764; 514/251; 424/692

(57) ABSTRACT

The present invention is directed to compositions, methods of delivery and packaged nutraceuticals of a stabilized Feverfew Extract. More specifically, the stabilized Feverfew extract contains at least about 4% parthenolide. In a particular embodiment, the stabilized Feverfew extract is provided in a soft gelatin capsule, at a concentration effective to provide relief of the identified affliction, such as a migraine headache.

### CROSS REFERENCE TO RELATED APPLICATION

This application claims benefit under 35 U.S.C. § 119(e) to application Ser. No. 60/547,528 (attorney docket 34095/US), entitled “Stabilized Feverfew Formulations” by Ronald Udell and Roy Bouskila on Feb. 25, 2004, the contents of which are incorporated herein in their entirety.

### FIELD OF THE INVENTION

The present invention relates to Feverfew formulations that contain efficacious amounts of stabilized parthenolides, thereby providing increased bioavailability in nutraceutical delivery.

### BACKGROUND OF THE INVENTION

Extracts of Tanacetum parthenium, a plant belonging to the family Asteracee/Composite, also known as Altamisa, Crisanthemum, Leucanthemum, Pyrethrum parthenium as well as under the common name “Feverfew”, have been used in the treatment of migraine, vertigo, arthritis, menstrual disorders, fever, toothache, stomach ache and insect bites. Extracts of Tanacetum parthenium contain various volatile oils having mono- and/or sesquiterpene components, flavonoids, tannins, and pyrethrin, as well as terpenoids of the family of sesquiterpene lactones known as germacranolides, guaianolides and eudesmanolides. These latter compounds are characterized by an alpha-unsaturated gamma-lactone structure (sequiterpene lactones, STLs). In particular the STLs include parthenolide, 3-beta-hydroxy-parthenoide, costunolide, 3-beta-hydroxy-costunolide, artemorin, 8-alpha-hydroxy-estafiatin and chrysanthemonin.

Feverfew is a short, bushy perennial plant that grows along fields and roadsides. The yellow-green leaves and yellow flowers of Feverfew resemble those of chamomile, for which it is sometimes confused. The flowers generally bloom from July to October. The leaves have been used in medicinal preparations. For example, Feverfew has enjoyed use by British herbalists as an analgesic in the treatment of fevers and arthritis, but had faded into obscurity until recently. Feverfew has enjoyed a revival over the past two decades due to approval of its use for treatment of migraine by both the Canadian and British governments.

Feverfew is rich in STLs, the most abundant of which, parthenolide, represents about 85% of the STL content in Feverfew and is the portion of the leaf believed to be responsible for Feverfew's anti-migraine activity. Parthenolide (1aR,4E,7aS,110aS,10bS)-2,3,6,7,7a,8,10a,10b-Octahydro-1a,5-dimethyl-8-methylene-oxireno[9,10]cyclodeca[1,2-b]furan-9(1aH)-one, depicted below includes an epoxide and a lactone ring:

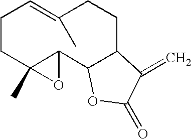

Feverfew extracts, containing parthenolides, have been isolated by various methods. However, the parthenolides, which are considered to be the active components of the extracts, are generally unstable and lose a portion of their nutraceutical activity within a year. This is possibly due to instability of the epoxide ring and/or from hydrolysis of the lactone ring. Additionally, common extraction techniques only provide Feverfew extracts that have about 1.0% (generally 0.7%) or less parthenolide(s) on a weight basis of total extract.

There is a need in the art for Feverfew compositions that provide stable parthenolide(s) and/or where the parthenolide(s) content is greater than about 1.0% of a weight basis of total extract. Additionally, there is a need to deliver such a Feverfew extract or Feverfew composition to an individual in need thereof to help alleviate varied ailments.

### BRIEF SUMMARY OF THE INVENTION

The present invention pertains to the surprising discovery that extraction of the active ingredients from Feverfew can be performed in a way to afford a Feverfew extract having a concentration of about 4.0% or greater parthenolide in the extract on a total weight basis. Up until the present invention, typical Feverfew extraction procedures yield less than 1% parthenolide based on total weight of a Feverfew extract. Extraction of Feverfew with acetone as described herein provides an extract having 4% parthenolide. A Feverfew extract containing 4% parthenolide is available from Galilee Herbal Remedies and as OptiPure Brand (Chemco Industries, Inc.) as “PharmaFew”.

The present invention more advantageously provides a “stabilized” Feverfew extract having at least about 4% parthenolide by weight of the total weight of the extract. The ability to stabilize the parthenolide in the Feverfew extract of the invention provides a material that has a shelf life of greater than 1 year, and more particularly at least about 3 years, without loss of nutraceutical activity of the parthenolide. Currently available Feverfew extracts degrade within about a year. Thus the potency of the parthenolide is compromised over time, and the individual using such a single source of unstabilized Feverfew extract(s)would not receive a uniform dosage as time progresses.

In one aspect, the invention pertains to dietary supplements, e.g., nutraceuticals, that include an extract from a Feverfew plant and magnesium wherein the Feverfew extract contains about 4.0% stabilized parthenolide. The magnesium is provided as magnesium oxide, magnesium chloride, magnesium citrate and the like.

In another aspect, the invention pertains to dietary supplements that include an extract from a Feverfew plant and magnesium wherein the Feverfew extract contains about 4.0% parthenolide stabilized with a stabilizing agent and the magnesium is provided as magnesium oxide, wherein the stabilizing agent is a salt of an organic acid having two or more carboxylic acid moieties. In one particular embodiment, the stabilizing agent is tricalcium citrate.

In still another aspect, the invention pertains to soft gelatin capsules that include an extract from a Feverfew plant wherein the Feverfew extract contains about 4.0% stabilized parthenolide. In particular, the stabilized parthenolide in the soft gelatin capsule is stabilized with a stabilizing agent comprising a salt of an organic acid having two or more carboxylic acid moieties, i.e., tricalcium citrate.

The various embodiments of the invention can contain, in addition to the stabilized parthenolide, various additives such as riboflavin, vitamin B2 and Feverfew powder.

The present invention further pertains to methods to treat or prevent various afflictions with the compositions of the invention. Such afflictions include inflammation, asthma, allergies, such as hay fever, migraine headaches vertigo, arthritis, menstrual disorders, fever, toothache, stomachache and insect bites.

The present invention also pertains to packaged nutraceutical compositions of the invention used to treat or prevent inflammation, asthma, allergies, such as hayfever and migraine headaches, as well as the remaining afflictions noted above.

In particular, the compositions of the invention are included in a soft gelatin (soft gel) capsule. Typically, the soft gelatin capsule includes at least about 25 mg to about 75 mg by weight of a Feverfew extract containing at least about 4% of parthenolide (PharmaFew) by total weight of the extract. The extract can be stabilized.

The number of dosages taken per day by the individual in need thereof controls the amount of parthenolide administered. For example, a soft gelatin capsule that contains about 40 mg of 4% parthenolide from a Feverfew extract, such as PharmaFew, can be taken once a day to prevent migraine headaches. Alternatively, two, three or more soft gelatin capsules that contain about 40 mg of 4% parthenolide from a Feverfew extract, such as PharmaFew, can be taken during a 24 hour period to treat an individual that is suffering with a migraine headache.

In another embodiment, a single dose of a soft gel capsule containing about 50 mg of 4% parthenolide Feverfew extract can be taken by an individual in need thereof for the treatment and/or prevention of arthritis. Alternatively, the dosage can be divided into multiple dosage to effect a sustained effect to the disease condition.

In still another embodiment, a single dose of a soft gel capsule containing about 50 mg of 4% parthenolide Feverfew extract can be taken by an individual in need thereof for the treatment and/or prevention of allergies, such as hay fever. Alternatively, the dosage can be divided into multiple dosage to effect a sustained effect to the allergy. Advantageously, the parthenolide containing compositions of the invention can be taken prophylactically prior to allergy season to prevent the onset of allergic reactions. The extracts noted throughout the specification, can all be stabilized.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### DETAILED DESCRIPTION

The present invention pertains to the surprising discovery that Feverfew extracts can be stabilized, such that the concentration of parthenolide, an active component of the Feverfew extract, does not degrade appreciably over up to a 3-year period. Up until the present discovery, typical Feverfew extracts lose approximately 15% of parthenolide stability during a period of one year. Thus, the present invention provides the ability to circumvent this decrease in potency and nutraceutical efficacy.

The herb, which is used in the present invention, is known correctly and variously as Chrysanthemum parthenium, Tanecetum parthenium and Matricania parthenium. All known plants having the characteristics, are therefore included in this application. Of these plants, Tanacetum parthenium (also known as Chrysanthenum parthenium and commonly known as Feverfew, featherfoil, flirtwort and Bachelor's Buttons) has been put forward as a herbal medicament or nutraceutical.

The extracts of Tanacetum parthenium that contain various volatile oils having mono- and/or sesquiterpene components, flavonoids, tannins, and pyrethrin, as well as terpenoids of the family of sesquiterpene lactones known as germacranolides, guaianolides and eudesmanolides are considered part of the invention. These latter compounds are characterized by an alpha-unsaturated gamma-lactone structure (sequiterpene lactones, STLs). In particular the STLs include parthenolide, 3-beta-hydroxy-parthenoide, costunolide, 3-beta-hydroxy-costunolide, artemorin, 8-alpha-hydroxy-estafiatin and chrysanthemonin, all of which are considered to be parthenolide analogs for the purposes of this invention.

Parthenolide is an active component of Feverfew extracts. Parthenolide, represents about 85% of the STL content in Feverfew and is the portion of the leaf believed to be responsible for Feverfew's anti-migraine and nutraceutical properties. Parthenolide (1aR,4E,7aS, 10aS,10bS)-2,3,6,7,7a,8,10a,10b-Octahydro-1a,5-dimethyl-8-methylene-oxireno[9,10]cyclodeca[1,2-b]furan-9(1aH)-one, depicted below, includes an epoxide and a lactone ring:

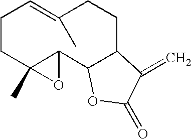

Feverfew extracts, containing parthenolides, have been isolated by various methods. However, the parthenolides, which are considered to be the active components of the extracts, are generally unstable and lose a portion of their nutraceutical activity within a year. This is possibly due to instability of the epoxide ring and/or from hydrolysis of the lactone ring. Additionally, common extraction techniques only provide Feverfew extracts that have about 1.0% (generally 0.7%) or less parthenolide(s) on a weight basis of total extract.

Various extraction procedures useful to isolate an extract that includes parthenolides include those described in U.S. Pat. Nos. 4,758,433, 5,384,121, 6,224,875, 6,479,080 and 6,23,768, the contents of which are incorporated herein by reference in their entirety. These extraction procedures generally require the use of a polar organic solvent for example acetonitrile, methanol, ethanol, isopropanol, ether, ethyl acetate, acetone or mixtures thereof. Ethanol is the preferred solvent, since it is the least toxic with regards to the residues being left in the final product.

Prior to the present invention, generally the mixture of plant tissue and polar organic solvent is left to stand to allow the extraction to take place. Alternatively, the plant tissue can be exhaustively extracted with a polar organic solvent in a Soxhlet apparatus or the like. The plant tissue, which is extracted in these known methods, may be fresh, frozen or dried and may be in comminuted form. The extract is then generally separated from the plant tissue and the solvent removed from the solvent extract by conventional techniques. Following removal of the solvent the remaining primary extract can be further purified by known techniques such as column chromatography and/or recrystallization and/or further solvent extraction. The remaining plant tissue may be further extracted using the same or an alternative solvent. These known extraction techniques provide a Feverfew extract that contains about 1% or less of parthenolide based on the total weight of the extract.

The present invention includes an extraction process that includes the steps as described below, whereby the Feverfew extract includes at least about a 4% parthenolide content by weight of total extract. The resultant extract is commercially available from Galilee Herbal Remedies (Israel) and as PharmaFew from Chemco Industries, Los Angeles, Calif., USA. The Feverfew extract contains at least about 4% parthenolide by weight of the total weight of the extract. The parthenolide extract is stabile for a period of at least one year and more particularly, at least about 3 years.

Not to be limited by theory, it is believed the Feverfew extract can be “stabilized” by treatment of the extract with an organic acid, in particular, an organic acid having two or more carboxylic acid moieties. In certain embodiments, the carboxylic acid is a salt. Suitable carboxylic acid containing moieties that are useful as “stabilizers” include, for example, adipic acid, pyruvic acid, gallic acid, tartaric acid and citric acid. Suitable salts include a metal ion, e.g., an alkali metal ion, an alkaline earth ion, or an aluminum or calcium ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19). In a particular embodiment, the salt of the carboxylic acid is tricalcium citrate. Generally, the ratio of stabilizer to FeverFew extract (on a dry weight basis) is in a range of between about 0.05 to 1 to about 1:1, more particularly from between about 0.2 to 1 to about 0.5 to 1, and also, 0.3 to 1 to about 0.6 to 1.

Pharmaceutically acceptable salts useful for stabilizing the Feverfew extract are those salts that retain substantially one or more of the desired pharmacological activities of the active component(s) and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the organic acid is either replaced by a metal ion (e.g., an alkali metal ion, an alkaline earth metal ion or an aluminum ion) or coordinates with an organic base (e.g., ethanolamine, diethanolamine, triethanolamine, N-methylglucamine, morpholine, piperidine, dimethylamine, diethylamine, etc.).

The extract can be treated with the stabilizing agent by many methods. For example, the stabilizing agent as a solid can be simply admixed with the dried or moist extract. Alternatively, the extract can be in solution and the stabilizing agent added as either a solid or as a solution. Removal of solvent and water from the mixture provides a material that is in part the stabilizing agent and the Feverfew extract. The stabilized material can be homogenous or heterogeneous. In one aspect, the Feverfew extract material is considered the be “coated”.

Coated Feverfew extract can be either a uniform coating, i.e., the Feverfew extract particles are considered to be encapsulated or the coating can be non-uniform. Non-uniform coatings are considered suitable when enough of the stabilizing agent is admixed with the Feverfew material, such that the Feverfew extract is stabilized, without loss of activity for at least one year.

The Feverfew extract useful in the invention is prepared by the following general isolation process. Approximately 1 kg of herbaceous material is treated with 2.5 kg acetone at a temperature between about 8 and 15° C. with agitation. The mixture is soaked/agitated for approximately 1.5 hours at this temperature, the solution is vacuum filtered, and the herbaceous material is again treated with 2.5 kg acetone. This sequence is repeated approximately three times, such that approximately 7.5 kg of acetone are used to isolate extracted material. The remaining herbaceous material is given a final rinse with 4 kg acetone and vacuum filtered.

The combined filtrates are then concentrated under reduced pressure to afford a thick paste. The paste is treated with a bulking agent, such as “β-CD” (beta-cyclodextrin) in water, and then a stabilizing agent, such as an organic acid or organic acid salt, such as calcium citrate acid is added. Approximately 0.25:1 tricalcium citrate is added to the extract on a weight to weight basis.

The resulting solution is spray dried to afford a stabilized Feverfew extract. The spray dried extract can be sieved with 60 mesh sieves, packaged and stored in aluminized bags, generally in 5 kg sizes.

Suitable bulking agents are known in the art and are defined as a compound useful in assisting redispersion of dried small particles back into a suspension or solution such as an aqueous suspension. Suitable bulking agents include hydroxyl-containing, hydrophilic, relatively low molecular weight (less than 50,000) compounds such as monosaccharides, disaccharides, trisaccharides, oligosaccharides, i.e., cyclodextrins, sucrose, raffinose, lactose, mannitol, sorbitol, trehalose, glycerol, dextrose, maltodextrose, fructose, sugars, pentoses, hexoses, xylitol, and mixtures thereof.

For the purposes of the invention, the term “cyclodextrin” is to be broadly construed and includes, without limitation, alpha-cyclodextrins, beta-cyclodextrins, and gamma-cyclodextrins. An example of a description of cyclodextrins is provided in The Merck Index, 12^{th} Ed., (p. 458) 1996. As known in the art cyclodextrins are cyclic oligosaccharides typically consisting of 6, 7, or 8 glucose units. The glucose units are linked by alpha-1,4-glucosidic bonds. As a consequence of the chair formation of the sugar units, all secondary hydroxyl groups (e.g., at C₂ and C₃) are located on one side of the ring, while all the primary hydroxyl groups at C₆ are situated on the other side. As a result, the external faces are hydrophilic, marking the cyclodextrins water-soluble. In contrast, the cavities of the cyclodextrins are hydrophobic, since they are lined by the hydrogen of atoms C₃, C₅, and by ether-like oxygens. Also encompassed under the definition of cyclodextrin are derivatives of cyclodextrins. In various embodiments, the 18 to 24 hydroxyl groups of the respective cyclodextrin molecules are the starting points for the synthesis of such derivatives. Using known techniques, methyl, ethyl-hydroxyethyl, hydroxymethyl, and hydroxypropyl substituted cyclodextrins may be utilized.

Examples of cyclodextrins that can be used in the present invention include, without limitation, hydropropyl-beta-cyclodextrin, hydroxyethylbeta-cyclodextrin, G2-alpha-cyclodextrin, G2-beta-cyclodextrin, gamma-cyclodextrin, and methyl-beta-cyclodextrin. A particularly preferred cyclodextrin is hydroxypropyl-beta-cyclodextrin (HP-β-CD). Combinations of cyclodextrins may also be employed for the purposes of the invention.

Optionally, bulking agents can include one or more amino acids, preferably naturally occurring or essential amino acids, proteins, peptides, vitamins such as vitamin A, vitamin C (ascorbic acid), citric acid, cellulose and modified cellulose acceptable for pharmaceutical/nutraceutial or food use such as carboxymethylcellulose and salts thereof, albumin, aspartame, povidone, crospovidone, croscarmellose sodium (Ac-Di-Sol) and related salts, additional phospholipids such as egg lecithin, magnesium salts such as magnesium stearate, magnesium carbonate, magnesium aluminum silicate, magnesium trisilicate, maltodextrin, polyethylene glycol, pluronic surfactants, polyethylene glycol esters acceptable for pharmaceutical/nutraceutical use, polyethylene glycol ethers acceptable for pharmaceutical/nutraceutical use, polymethacrylates acceptable for pharmaceutical/nutraceutical use, polyvinyl alcohol, polyvinyl acetate and partially hydrolyzed polyvinyl acetate, saccharin, sodium saccharin, potassium sorbate, silicon dioxide, sodium lauryl sulfate, sorbitol, starch and modified starch, acceptable organic acids such as stearic acid, palmitic acid, tartaric acid, sorbic acid, fumaric acid, alginic acid, lactic acid, edetic acid, and acceptable salts thereof, flavorings, coloring agents, and other excipients such as diglycerides and triglycerides, fatty acids such as oleic acid, stearic acid, palmitic acid, and myristic acid, fatty acid sorbitan esters, Tween surfactants, PEG-castor oil surfactants, omega-3-fatty acids and their salts, and mixtures thereof.

Suitable spray drying techniques include those known in the art where a solution is formed into finely dispersed droplets that are dried in a medium, such as air, in a short retention time of approximately 1 to 45 seconds. The solution can be formed into fine droplets by various droplet forming apparatus', such as by centrifugal wheel atomizers, rotary wheel atomizers, pressure nozzle atomizers and the like. The solution can be evaporated by parallel flow, mixed flow or counter current flow of a gaseous stream. The spray drying environment can be heated or the process can be conducted at room temperature.

Spray drying of the solution with or without bulking agent can be accomplished using a commercially available spray drying apparatus such as a LabPlant SD05 Spray Dryer or using a larger scale spray drying apparatus. Dry air or dry oxygen free air or nitrogen or other non-oxidizing non-reactive dry gas is used in the spray drying. Moisture level in the isolated spray dried powder obtained as an initial product in the spray drying process is preferably below 3%, more preferably below.

The present invention provides one or more of the advantages where the Feverfew extract is in a concentration more than twice that what is typically commercially available; the Feverfew extract is stabilized, such that the parthenolide concentration remains at about 4% or greater for a period of at least a year, in particular 3 years; and the Feverfew extract (with the parthenolide) is administerable in a soft gel capsule. Soft gelatin capsules provide ease in swallowing which is beneficial for many individuals who have a difficult time swallowing pills, tablets and the like.

It should be understood, that throughout the specification, reference is made to parthenolide, and that such reference includes the analogs thereof.

The phrase “sufficient quantity of a stabilizing agent suitable to stabilize the Feverfew extract” is intended to mean that that amount of an organic dicarboxylic acid, in particular a salt, that will interact with the Feverfew extract, such that the extract does not lose potency of the parthenolide constituents over at least a one year period. This determination should be understood by one skilled in the art and can be determined by methods known in the art, such as by extraction, HPLC, GC, NMR or IR studies.

Formulation of the Feverfew extract and/or additional active ingredients can be accomplished by many methods known in the art. For example, the stabilized Feverfew is formulated in a suspension, an emulsion, an elixir, a solution, a caplet that harbors the liquid, or in a soft gelatin capsule. Often the formulation will include an acceptable carrier, such as an oil, or other suspending agent.

Suitable carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The formulations of the invention are considered dietary supplements useful to increase the amounts of Feverfew active components, such as the parthenolides, in the individuals in need thereof.

Alternatively, the formulations of the invention are also considered to be nutraceuticals. The term “nutraceutical” is recognized in the art and is intended to describe specific chemical compounds found in foods that may prevent disease. Feverfew extracts are considered to be such compounds.

The formulations of the invention can include dried “Feverfew” compositions, which are dried leaves of the Feverfew containing plants. The combination of such ingredients is know in the art and is considered “full spectrum” applications. Such mixtures of dried Feverfew and the stabilized Feverfew of the invention are within the scope and teachings of the present invention.

As previously discussed, the stabilized Feverfew extract of the invention can treat or prevent one or more of the afflictions discussed throughout the present specification. As a consequence of these activities, the stabilized Feverfew extracts of the invention can be used in a variety of in vitro, in vivo and ex vivo contexts to regulate or inhibit the afflictions described.

The formulations of the invention can further include various ingredients to help stabilize, or help promote the bioavailability of the Feverfew extract (and in particular the parthenolide(s)), or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, phospholipids, L-camitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Compositions of the invention, including soft gelatin capsules, can include, in addition to the sesquiterpene lactones, other known anti-migraine preparations, analgesics and antiemetics such as propranolol hydrochloride, ergotamine, methysergide, dihydroergotamine, clonidine hydrochloride, isometheptene mucate, buclizine dihydrochloride, metoclopramide hydrochloride, pizotifen, aspirin and other non-steroidal anti-inflammatory agents, paracetamol, ginko biloba, ergotamine, dihydroergotamine, isometheptene mucate, other analgesics (non-opoid analgesics, opoid analgesics or compound analgesic preparations) and/or anti-emetics (hyoscine, anti-histamines, phenothiazines, metoclopramide or clomperidone), racetamol and other minor analgesics, pentazocine hydrochloride, prochlorperazine, caffeine, meprobamate, ethoheptazine citrate, zomepirac and/or meptazinol hydrochloride.

Compositions of the invention, including soft gelatin capsules, can include non-steroidal antinflammatory drugs, analgesics, skeletal muscle relaxants, steroids, gold, penicillamine, aspirin, indomethacin, piroxicam, benorylate, ibuprofen, paracetamol, salicylamine, diflunisal, ethoheptazine, fenoprofen(calcium fenoprofate), flufenamic acid, mefenamic acid, naproxen sodium, ketoprofen, phenylbutazone, sulindac, penicillamine, salsalate, fenclofenac, flurbiprofen, fenbufen, feprazone, sodium aurothiomalate, naproxen, benoxaprofen, aloxiprin, hydroxychloroquine sulphate, azapropazone, oxyphenbutazone, tolemtin, choline magnesium trisalicylate, diclofenac, and/or adrenal steroids such as prednisone or prednisolone, systemic corticosteroids and corticotrophin, anti-malarials, immuno-suppressants and sulphasalazine.

Compositions of the invention, including soft gelatin capsules, can include a bronchodilator, antihistamine and/or anti-infective agents, examples of which include isoprenaline sulphate, orciprenaline, adrenaline, terbutaline sulphate, theophylline, choline theophyllinate, aminophylline, ephedrine hydrochloride, papaverine hydrochloride, ipratropium bromide, atropine methonitrate, beclomethasone dipropionate, fenoterol hydrobromide, betamethasone, isoetharine mesylate or hydrochloride, phenylephrine hydrochloride or bitartrate, thenyldiamine hydrochloride, reproterol hydrochloride, deptropine citrate, butethamate citrate, acepifylline, dipheylpyraline hydrochloride, sodium cromoglycate, etamiphylline camsylate, theophylline monoethanolamine, etafedrine hydrochloride, bufylline, guaiphenesin, diphenydramine hydrochloride and other histamine H₁-receptor antagonists, diprophylline, methoxyphenamine hydrochloride, rimiterol hydrobromide, hyoscine hydrobromide, salbutamol sulphate, ketotifen hydrogen fumarate, pseudo-ephedrine hydrochloride, bromhexine hydrochloride, adrenoceptor stimulants, anti-muscarinic bronchodilators, or compound bronchodilator preparations), anti-histamines(promethazine, trimeprazine, dimenhydrinate, chlorpheniramine, cyclizine, mequitazine, acrivastine, astemizole, cinnarizine, loratadine or terfenadine or anti-infective agents (anti-fungal, anti-vital or anti-bacterial agents).

Any dosage form, and combinations thereof, are contemplated by the present invention. Examples of such dosage forms include, without limitation, chewable tablets, elixirs, liquids, solutions, suspensions, emulsions, capsules, soft gelatin capsules, hard gelatin capsules, caplets, lozenges, chewable lozenges, suppositories, creams, topicals, ingestibles, injectables, infusions, health bars, confections, animal feeds, cereals, cereal coatings, and combinations thereof. The preparation of the above dosage forms are well known to persons of ordinary skill in the art.

For example, health bars can be prepared, without limitation, by mixing the formulation plus excipients (e.g., binders, fillers, flavors, colors, etc.) to a plastic mass consistency. The mass is then either extended or molded to form “candy bar” shapes that are then dried or allowed to solidify to form the final product.

Soft gel or soft gelatin capsules can be prepared, for example, without limitation, by dispersing the formulation in an appropriate vehicle (e.g. rice bran oil and/or beeswax) to form a high viscosity mixture. This mixture is then encapsulated with a gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams. In particular, the soft gel capsule typically weighs between about 1000 milligrams and 1300 milligrams, wherein the percentage fill is about 50% of the entire weight of the capsule, i.e., from about 500 to about 650 milligrams fill weight. The fill weight includes the active ingredient(s), solubilizing agents, etc.

For example, when preparing soft gelatin shells, the shell can include between about 20 to 70 percent gelatin, generally a plasticizer and about 5 to about 60% by weight sorbitol. The filling of the soft gelatin capsule is liquid (principally rice bran oil and/or beeswax if desired) and can include, apart form the antioxidant actives, a hydrophilic matrix. The hydrophilic matrix, if present, is a polyethylene glycol having an average molecular weight of from about 200 to 1000. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

In one embodiment, the gelatin used to prepare the soft gelatin capsule includes gelatin from lime or acid derived gel manufacturing processes known in the art. The gelatin is combined with plasticizers, such as glycerin, sorbitol or other polyalcoholic compounds, or combinations thereof and purified water. Optional additives can include colorants, preservatives, flavors, sweetening agents and/or opacifying agents. The amount of gelatin in the mixture can range from about 30 to about 60 percent (by weight), with about 15 to about 55% plasticizer (by weight) and purified water from about 15 to about 40% by weight. Optional additives are generally present in a range from about 0.1 to about 15% by weight.

A soft gel capsule is prepared by mixing the parthenolide and optional components, for a period of time until the mixture is thoroughly mixed, optionally under vacuum. A gelatin mixture is fed into two spreader boxes, which in turn form two gelatin ribbons that are used to make each half of the gelatin capsule shell. The fill mixture (parthenolide extract, bees wax and wheat germ oil, as an example) is pumped into the gelatin ribbons held in place by two rotating die cavity rolls. The capsules are half sealed when a pump injects the fill material into the die cavities. The injection is followed by forming hermetic seals between the two capsule halves and the capsules are cut from the gelatin ribbon.

Typically, when a soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams.

For example, a soft gel capsule can be prepared by mixing about 60 to about 75 grams of a stabilized Feverfew extract having about a 4% parthenolide content by weight of total extract, with between about 200 grams and about 250 grams (e.g., 225 grams) rice bran or soybean oil. The mixture can further include yellow bees wax. The mixture is then encapsulated within a gelatin capsule as described above.

The present invention also provides packaged formulations of a stabilized Feverfew extract and instructions for use of the tablet, capsule, elixir, etc. Typically, the packaged formulation, in whatever form, is administered to an individual in need thereof that requires an increase in the amount of the components in the individual's diet. Typically, the dosage requirement is between about 1 to about 4 dosages a day.

Feverfew, and specifically parthenolide, has been implicated in the inhibition of platelet aggregation and histamine release. It has also been shown to inhibit release of serotonin from platelets and polymorphonuclear leukocyte granules. Not to be limited by theory, this is believed to reduce the severity, duration and frequency of migraine headaches and lead to an improvement in blood vessel tone. Feverfew also inhibits prostaglandin synthesis and the release of arachidonic acid. This action may explain its historical use for inflammatory conditions such as arthritis.

Additional embodiments of the invention include Feverfew compositions that include, for example, magnesium, white willow bark, ginger, kava, and vitamin B. In another embodiment, the Feverfew composition can include devils claw, ginger, vitamin E and glucosamine. In still another embodiment, the Feverfew composition can include nettle and vitamin C. In still yet another embodiment, the Feverfew composition can include coral calcium. In yet another embodiment the Feverfew composition can include Echinacea, vitamin C or quercitin.

As previously discussed, the stabilized Feverfew extract of the invention can treat or prevent one or more of the afflictions discussed throughout the present specification. As a consequence of these activities, the stabilized Feverfew extracts of the invention can be used in a variety of in vitro, in vivo and ex vivo contexts to regulate or inhibit the afflictions described.

When used to treat or prevent such diseases, the stabilized Feverfew extracts can be administered singly, as mixtures of one or more active compounds or in mixture or combination with other agents useful for treating such diseases and/or the symptoms associated with such diseases. The Feverfew extracts can also be administered in mixture or in combination with agents useful to treat other disorders or maladies, such as steroids, and antihistamines, to name a few.

Nutraceutical or pharmaceutical compositions comprising the Feverfew extracts of the invention cay be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries, which facilitate processing of the active compounds into preparations, which can be used pharmaceutically.

Pharmaceutical or nutraceutical compositions of the invention can take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the Feverfew extracts of the invention can be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the Feverfew extract can dried by any art-known technique, such as lyophilization, and reconstituted prior to use.

For transmucosal administration, penetrants appropriate to the barrier to be permeated are used in the formulation. Such penetrants are known in the art.

For oral administration, the compositions can take the form of, for example, lozenges, tablets or capsules prepared by conventional means with acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars or enteric coatings.

Liquid preparations for oral administration can take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration can be suitably formulated to give controlled release of the Feverfew extract, as are well known.

For buccal administration, the compositions can take the form of tablets or lozenges formulated in conventional manner.

For rectal and vaginal routes of administration, the Feverfew extract can be formulated as solutions (for retention enemas) suppositories or ointments containing conventional suppository bases such as cocoa butter or other glycerides.

For nasal administration or administration by inhalation or insufflation, the Feverfew extracts can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For prolonged delivery, the stabilized Feverfew extracts of the invention can be formulated as a depot preparation for administration by implantation or intramuscular injection. The Feverfew extract can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the active compound(s) for percutaneous absorption, may be used. To this end, permeation enhancers can be used to facilitate transdermal penetration of the active compound(s). Suitable transdermal patches are described in for example, U.S. Pat. No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical and nutraceutical delivery systems can be employed. Liposomes and emulsions are well-known examples of delivery vehicles that may be used to deliver the Feverfew extract. Certain organic solvents such as dimethylsulfoxide (DMSO) may also be employed, although usually at the cost of greater toxicity.

The compositions can, if desired, be presented in a pack or dispenser device, which can contain one or more unit dosage forms containing the Feverfew Extract. The pack can, for example, comprise metal or plastic foil, such as a blister pack. The pack or dispenser device can be accompanied by instructions for administration.

The nutraceutical compositions of the invention, will generally be used in an amount effective to achieve the intended result, for example in an amount effective to treat or prevent the particular affliction being treated. The nutraceutical can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the individual reports an improvement in feeling or condition, notwithstanding that the individual may still be afflicted with the underlying affliction. For example, administration of a nutraceutical to an individual suffering from an allergy provides therapeutic benefit not only when the underlying allergic response is eradicated or ameliorated, but also when the individual reports a decrease in the severity or duration of the symptoms associated with the allergy following exposure to the allergen. As another example, therapeutic benefit in the context of asthma includes an improvement in respiration following the onset of an asthmatic attack, or a reduction in the frequency or severity of asthmatic episodes. Therapeutic benefit also includes halting or slowing the progression of the disease, regardless of whether improvement is realized.

For prophylactic administration, the nutraceutical can be administered to an individual at risk of developing one of the previously described conditions. For example, if it is unknown whether an individual is allergic to a particular drug, the nutraceutical can be administered prior to administration of the drug to avoid or ameliorate an allergic response to the drug. Alternatively, prophylactic administration can be applied to avoid the onset of symptoms in an individual diagnosed with the underlying disorder. For example, a nutraceutical may be administered to an allergy sufferer prior to expected exposure to the allergen. Nutraceuticals may also be administered prophylactically to healthy individuals who are repeatedly exposed to agents known to one of the above-described maladies to prevent the onset of the disorder. For example, a nutraceutical may be administered to a healthy individual who is repeatedly exposed to an allergen known to induce allergies, such as latex, in an effort to prevent the individual from developing an allergy. Alternatively, a nutraceutical may be administered to an individual suffering from asthma prior to partaking in activities, which trigger asthma attacks to lessen the severity of, or avoid altogether, an asthmatic episode.

The amount of nutraceutical administered will depend upon a variety of factors, including, for example, the particular indication being treated, the mode of administration, whether the desired benefit is prophylactic or therapeutic, the severity of the indication being treated and the age and weight of the individual, the bioavailability of the particular active compound, etc. Determination of an effective dosage is well within the capabilities of those skilled in the art.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pagamonon Press, and the references cited therein.

Initial dosages can also be estimated from in vivo data, such as animal models. Animal models useful for testing the efficacy of compounds to treat or prevent the various diseases described above are well-known in the art. Suitable animal models of hypersensitivity or allergic reactions are described in Foster, 1995, Allergy 50(21Suppl):6-9, discussion 34-38 and Tumas et al., 2001, J. Allergy Clin. Immunol. 107(6):1025-1033. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but may be higher or lower, depending upon, among other factors, the activity of the, its bioavailability, the mode of administration and various factors discussed above. Dosage amount and interval may be adjusted individually to provide plasma levels of the nutraceutical, which are sufficient to maintain therapeutic or prophylactic effect. In cases of local administration or selective uptake, such as local topical administration, the effective local concentration of active compound(s) may not be related to plasma concentration. Skilled artisans will be able to optimize effective local dosages without undue experimentation.

The nutraceutical composition can be administered once per day, a few or several times per day, or even multiple times per day, depending upon, among other things, the indication being treated and the judgment of the individual and by general directions provided by the packaged formulation.

The following examples are intended to be illustrative only and should not be considered limiting.

### EXAMPLES

Formulations of a stabilized Feverfew extract can be prepared by the following ratios by mixing the components together and then placing into a soft gel capsule.

| Component | Example 1 |
|---|---|
| PharmaFew (Feverfew 4% active stabile parthenolide) | 30 mg |
| Lecithin | 8 mg |
| Soybean Oil | 102 mg |
| Yellow Bees Wax | 10 mg |
| Total weight | 150 mg |

The resultant mixture of the above-identified components provides a fluid suspension that is encapsulated in soft gel capsules. The “fill weight” of 150 mg can be encapsulated to afford a soft gel capsule having a total weight of between about 500 mg and 1000 mg, depending upon the die size used to prepare the soft gel capsule.

Preparation of the soft gel capsules was accomplished by methods well known in the art including, but not limited to, those described throughout the specification and in U.S. Pat. Nos. 6,616,942, 6,623,734 and pending U.S. Ser. Nos. 10/035,753 and 09/825,920, the contents of which are incorporated herein by reference in their entirety.

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

1. A dietary supplement comprising, an extract from a Feverfew plant and magnesium wherein the Feverfew extract contains about 4.0% stabilized parthenolide and the magnesium is provided as magnesium oxide.

2. A dietary supplement comprising, an extract from a Feverfew plant and magnesium wherein the Feverfew extract contains about 4.0% parthenolide stabilized with a stabilizing agent and the magnesium is provided as magnesium, wherein the stabilizing agent is a salt of an organic acid having two or more carboxylic acid moieties.

3. The dietary supplement of claim 2 wherein the organic acid salt is tricalcium citrate.

4. A soft gelatin capsule comprising an extract from a Feverfew plant wherein the Feverfew extract contains about 4.0% stabilized parthenolide.

5. The soft gelatin capsule of claim 4, further comprising a magnesium compound.

6. The soft gelatin capsule of claim 5, wherein the magnesium compound is magnesium oxide.

7. The soft gelatin capsule of claims 5, further comprising riboflavin.

8. The soft gelatin capsule of claim 5, further comprising vitamin B2.

9. The soft gelatin capsule of claim 5, further comprising Feverfew powder.

10. The soft gelatin capsule of claim 5, wherein the stabilized parthenolide is stabilized with a stabilizing agent comprising a salt of an organic acid having two or more carboxylic acid moieties.

11. The soft gelatin capsule of claim 10, wherein the stabilizing agent is tricalcium citrate.

12. A method to treat or prevent inflammation, asthma, hayfever or migraine headaches, comprising administering an effective amount of a dietary supplement comprising, an extract from a Feverfew plant and magnesium wherein the Feverfew extract contains about 4.0% stabilized parthenolide and the magnesium is provided as magnesium oxide, such that the inflammation, asthma, hayfever or migraine headache is treated or prevented.

13. A method to treat or prevent inflammation, asthma, hayfever or migraine headaches, comprising administering an effective amount of a dietary supplement comprising, an extract from a Feverfew plant and magnesium wherein the Feverfew extract contains about 4.0% parthenolide stabilized with a stabilizing agent and the magnesium is provided as magnesium oxide, wherein the stabilizing agent is a salt of an organic acid having two or more carboxylic acid moieties, such that the inflammation, asthma, hayfever or migraine headache is treated or prevented

14. A method to treat or prevent inflammation, asthma, hayfever or migraine headaches, comprising administering an effective amount of a soft gelatin capsule comprising an extract from a Feverfew plant wherein the Feverfew extract contains about 4.0% stabilized parthenolide.

15. The method of claim 14, wherein the stabilized parthenolide is stabilized with a stabilizing agent comprising a salt of an organic acid having two or more carboxylic acid moieties.

16. The method of claim 15, wherein the stabilizing agent is tricalcium citrate.

17. A packaged nutraceutical for treating or preventing inflammation, asthma, hayfever or migraine headaches comprising:
an effective amount of a nutraceutical supplement comprising, an extract from a Feverfew plant and magnesium wherein the Feverfew extract contains about 4.0% stabilized parthenolide and the magnesium is provided as magnesium oxide; and
instructions for administration of the dietary supplement to treat or prevent inflammation, asthma, hayfever or migraine headaches.

18. A packaged nutraceutical for treating or preventing inflammation, asthma, hayfever or migraine headaches comprising:
an effective amount of a nutraceutical supplement comprising, an extract from a Feverfew plant and magnesium wherein the Feverfew extract contains about 4.0% parthenolide stabilized with a stabilizing agent and the magnesium is provided as magnesium oxide, wherein the stabilizing agent is a salt of an organic acid having two or more carboxylic acid moieties; and
instructions for administration of the dietary supplement to treat or prevent inflammation, asthma, hayfever or migraine headaches.

19. A packaged nutraceutical for treating or preventing inflammation, asthma, hay fever or migraine headaches comprising:
an effective amount of a nutraceutical supplement comprising a soft gelatin capsule comprising an extract from a Feverfew plant wherein the Feverfew extract contains about 4.0% stabilized parthenolide; and
instructions for administration of the dietary supplement to treat or prevent inflammation, asthma, hay fever or migraine headaches.

20. The packaged nutraceutical of claim 19, wherein the stabilized parthenolide is stabilized with a stabilizing agent comprising a salt of an organic acid having two or more carboxylic acid moieties.
